(19) United States
(12) Patent Application Publication
(10) Pub. No.: US 2010/0092402 A1
(43) Pub. Date: Apr. 15, 2010

(54) TREATMENT OF RESPIRATORY DISEASE

(75) Inventors: Roderick Lindsay Hall, London (GB); Peter John Cole, London (GB)

Correspondence Address: RATNERPRESTIA, P.O. BOX 980, VALLEY FORGE, PA, 19482, US

(73) Assignee: Mucokinetica Ltd., London (GB)

(21) Appl. No.: 12/524,506

(22) PCT Filed: Jan. 25, 2008
(86) PCT No.: PCT/GB08/00277

§ 371 Date: Dec. 11, 2009

(30) Foreign Application Priority Data
Jan. 25, 2007 (GB) 0701456.6

(51) Int. Cl. A61K 31/24; A61K 39/00; A61P 11/00; A61K 9/12; C07C 257/18

(52) U.S. Cl. 424/45; 514/540; 424/184.1; 424/43; 560/60

(57) ABSTRACT

The invention relates to the use of amidino compounds in the manufacture and formulation of medicaments for the treatment of respiratory diseases.

### FIELD OF INVENTION

The current invention relates to a new medical use of a family of amidino compounds in the treatment of respiratory diseases.

In a preferred embodiment the invention relates to the use of 6-amidino-2-napthyl 4-guanidinobenzoate in the manufacture of a medicament for the treatment of respiratory disease.

### BACKGROUND

In the respiratory epithelium the properties and composition of the airway surface liquid (ASL) that lines the airway surfaces are critical to effective operation of airway defence functions, especially mucociliary clearance. Absorption of sodium ions via amiloride-sensitive sodium channels (ENaC) located at the apical surface of epithelial cells is particularly important in regulating mucociliary function in health and disease. Pharmacological treatments applied to the airway epithelium to lower ENaC activity also act to increase mucociliary clearance activity. Mucostasis in respiratory disease (i.e. accumulation of secretions and defective clearance) is an important untreated medical need. Poor clearance of secretions from the lower airways is a continuing problem and is believed to lead to chronic infection, inflammation in the airways, reduction in lung function and in some conditions to destruction of the lungs and eventually to respiratory failure. Lowering ENaC activity is a promising strategy to improve performance of mucociliary clearance in respiratory diseases characterised by mucostasis; cystic fibrosis (CF); bronchiectasis; chronic bronchitis; chronic obstructive pulmonary disease (COPD); rhino-sinusitis; otitis media. An effective treatment to enhance mucociliary clearance is thought to have potential to achieve significant benefit for respiratory disease including diseases of the lung, airways (bronchioles, bronchi), and upper respiratory tract (nose, para-nasal sinuses, Eustachian tube and middle ear).

Amiloride and analogues have been evaluated in the clinic as potential inhaled therapies for treatment of respiratory diseases characterised by mucostasis, particularly CF. Although amiloride is a potent blocker of ENaC the clinical results have been disappointing. Rapid reversibility of binding to ENaC together with rapid absorption by the airway epithelium are thought to be responsible for the short duration of efficacy achieved in patients.

Amiloride is a potent, but short acting blocker of the epithelial sodium channel, where reduction of ENaC activity is achieved by direct exofacial block in the pore of the channel. Washing epithelial cells to remove amiloride results in rapid recovery of ENaC activity, typically within 1 to 2 minutes.

Down regulation of airway ENaC activity has also been achieved by Kunitz-type serine protease inhibitors, aprotinin and BAY 39-9437 (Bridges et al 2001). The proposed mechanism of action of the Kunitz inhibitors is to inhibit (an) endogenous protease(s) responsible for proteolytic activation of sodium channel proteins in the apical membrane of epithelial cells. By contrast to the short acting blocker profile of amiloride, BAY 39-9437 is an example of a long duration of action down regulator of ENaC. Treatment of airway epithelial cells with BAY 39-9437 results in reduction of ENaC activity that is sustained for an extended period of time after unbound substance has been removed, by washing, from contact with the epithelial cells (Bridges et al 2001).

Potent, long duration down regulators of ENaC activity are potentially efficacious inhaled treatments for respiratory diseases characterised by mucostasis.

6-amidino-2-napthyl 4-guanidinobenzoate, was first described in Japanese patent no. JP57053454. It is also described in United States patent no. U.S. Pat. No. 4,454,338 as a member of a family of related amidino compounds, which are described as having powerful antitrypsin, antiplasmin, antikallikrein, antithrombin, antigranzyme and anticompliment activity. 6-amidino-2-napthyl 4-guanidinobenzoate is also known to act as an anti-inflammatory agent (Iwaki et al 1984) and an anticoagulant (Hitomi et al 1985).

The substance has been approved for use in the treatment of the acute symptoms of pancreatitis, disseminated intravascular coagulation and patients with bleeding complications or tendency to bleed. It has also been used to prevent blood coagulation during extracorporeal blood circulation, for example in hemodialysis and plasmapheresis.

Muto et al. (1993) reported that 6-amidino-2-napthyl 4-guanidinobenzoate perfused at a high dose of 10⁻⁴ M in the cortical collecting duct of the rabbit kidney caused a change in transepithelial voltage equivalent to 40% of that achievable by 5.10⁻⁶ M amiloride. The 10-fold lower concentration of 10⁻⁵ M 6-amidino-2-napthyl 4-guanidinobenzoate was without effect on the rabbit kidney model. It should be noted that the minimum effective concentration in the kidney model is 1000-fold higher than the minimum significant concentration that achieves down regulation of ENaC activity in the human airway epithelial cell.

The profile of the change in transepithelial voltage in response to perfusion of 10⁻⁴ M 6-amidino-2-napthyl 4-guanidinobenzoate (Muto at al. 1993) shows rapid onset within 30s of perfusion, and rapid recovery within 1 minute to starting voltage when perfusion is stopped. This profile is similar to that observed for perfusion of amiloride in the model. It is known that after intravenous infusion of 6-amidino-2-napthyl 4-guanidinobenzoate in animals and man that the metabolites 4-guanidinobenzoic acid (pGBA) and amidino-2-napthol (AN) are excreted into the urine via the kidney cortical collecting duct.

In a later paper (Muto et al. 1994) the same authors investigated the possibility that the observed effects in the kidney model could be achieved by the metabolites. They reported (Muto at al. 1994) that pGBA was active when perfused at 10⁻⁵ M, 10-fold lower than the minimum effective concentration for 6-amidino-2-napthyl 4-guanidinobenzoate. AN was also active but the minimum effective concentration was again 10⁻⁴ M (Muto et al. 1994). Muto et al. (1993; 1994) report that in the kidney model 6-amidino-2-napthyl 4-guanidinobenzoate may act as a weak, short acting inhibitor of the kidney sodium channel, and that the effect may be mediated by the metabolites of 6-amidino-2-napthyl 4-guanidinobenzoate, particularly pGBA that was 10-fold higher potency than 6-amidino-2-napthyl 4-guanidinobenzoate. The kidney model results do not predict potent, long duration down regulation of airway ENaC activity sustained at least 1 h 45 min. after washout from contact with doses up to 1000-fold below the minimum effective concentration in the kidney model.

Rossier (2004) reviewed hormonal and serine protease regulation of the epithelial sodium channel. There are clear differences in hormonal control of the activity of ENaC in different organs and tissues. In the kidney and colon ENaC expression is under mineralocorticoid (aldosterone) control, whereas in the distal lung glucocorticoid control operates.

The first report (Vallet et al. 1997) of a serine protease activating ENaC was channel activating protease 1 (CAP-1) in Xenopus oocytes—an amphibian egg model. Since the first report at least two further channel activating proteases (CAP-2 & 3) have been reported with homologues in mammalian species, including human (see Rossier, 2004). Involvement of CAP homologues in regulation of ENaC activity in mammalian epithelial systems, including the kidney and the airway, has been reported. It is currently not known if regulation of ENaC activity in mammalian systems is controlled by activity of individual CAP homologues, or by mechanisms involving a combination of two, or a cascade of all three.

The mammalian homologue of CAP-1 is prostasin. Prostasin expressed in the membrane of human airway epithelial cells has been implicated (Tong et al. 2004) to have an involvement in protease regulation of ENaC in airway epithelial cells. The contribution of homologues of the other CAPs, or other serine proteases to the normal and pathological regulation of ENaC in the airways is not known at this time. There are no reports that 6-amidino-2-napthyl 4-guanidinobenzoate is an inhibitor of the enzymic catalytic activity of CAPs or CAP homologues, including prostasin.

Differences have been reported in the details of the protease regulation mechanisms in different mammalian epithelia and organs. Consistent with mineralocorticoid regulation of ENaC expression in the kidney, Narikiyo et al. (2002) found that aldosterone also increased prostasin expression in the mouse kidney cell line M-1, and also that urinary excretion of prostasin was increased approximately 4-fold when rats were intravenously infused with aldosterone.

Differences in cellular expression of prostasin have also been noted. Full length prostasin is a glycosylphosphatidylinositol (GPI)-membrane anchored protein. Tong et al. (2004) reported that in three human airway epithelial cell lines, JME/CF15, Calu-3, and A549, prostasin was expressed as a membrane anchored protein with little secreted into the medium in which the cells were cultured. By contrast Iwashita et al. (2003) observed that for the mouse kidney cell line, M-1 cells, prostasin was found secreted in the culture medium, and not in the membrane fraction of the cells as GPI-membrane anchored prostasin, nor was prostasin detected in the cell cytosol fraction. Iwashita et al. (2003) also reported detection of prostasin in the urine of rats and humans, and being unable to identify prostasin in cytosolic or membrane fractions prepared from rat kidneys. They postulate that in both M-1 cells and in the kidneys prostasin is expressed as a secretory protein, rather than as a membrane bound protein as observed for airway cells.

Iwashita et al. (2003) reported that treatment of M-1 kidney cells with 6-amidino-2-napthyl 4-guanidinobenzoate for 24 h reduced prostasin secretion into culture medium; also rats infused i.v. with 6-amidino-2-napthyl 4-guanidinobenzoate for 24h showed reduced levels of prostasin secreted in the urine. However, given the clear protein expression differences for prostasin between airway epithelial cells and kidney, it is not apparent how, or if, 6-amidino-2-napthyl 4-guanidinobenzoate would affect prostasin expression or ENaC activity in airway epithelial systems, and at time periods much shorter than the 24 h studied in the kidney models.

The current inventors are therefore unaware of any previous use of 6-amidino-2-napthyl 4-guanidinobenzoate or related compounds to regulate the activity of ENaC in airway epithelial cells or the respiratory system in general.

Surprisingly, the current inventors have found 6-amidino-2-napthyl 4-guanidinobenzoate to be a potent long duration inhibitor of ENaC activity in airway epithelial cells. This and related compounds therefore have potential in the treatment of respiratory diseases characterised by mucostasis such as cystic fibrosis (CF); bronchiectasis; chronic bronchitis; chronic obstructive pulmonary disease (COPD); rhino-sinusitis and otitis media.

### STATEMENT OF INVENTION

In a first aspect the present invention is directed to the use of an amidino compound of the general formula (I):

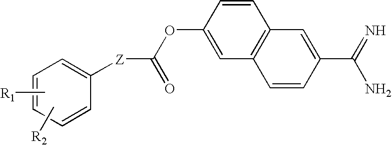

in the manufacture of a medicament for the treatment of respiratory disease.

In formula (I), Z represents —(CH₂)ₐ—,

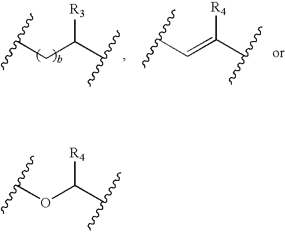

wherein a is 0, 1, 2 or 3, b is 0,1 or 2, R₃ is a straight or branched chain alkyl group of 1 to 4 carbon atoms or a cycloalkyl group of 3 to 6 carbon atoms, R₄ is a hydrogen atom or a straight or branched chain alkyl group of 1 to 4 carbon atoms.

R₁ and R₂, which may be the same or different, represent each a hydrogen atom, a straight or branched chain alkyl group of 1 to 4 carbon atoms, —O—R₅, —S—R₅, −COOR₅, —COR₆, —O—COR₇, —NHCOR₇,

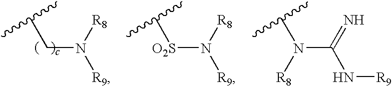

NO₂, CN, halogen, CF₃, methylenedioxy, or

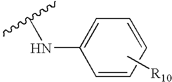

wherein c is 0, 1 or 2; R₅ is a hydrogen atom, linear or branched chain alkyl group of 1 to 4 carbon atoms, or benzyl group; R₆ is a hydrogen atom or straight or branched chain alkyl group of 1 to 4 carbon atoms; R₇ is a straight or branched chain alkyl group of 1 to 4 carbon atoms; R₈ and R₉, which may be the same or different, are each a hydrogen atom, straight or branched chain alkyl group of 1 to 4 carbon atoms, or amino radical protecting group; and R₁₀ is a hydrogen atom, methyl or CF₃. The straight or branched chain alkyl groups of 1 to 4 carbon atom include methyl, ethyl, n-propyl, isopropyl, n-butyl, isobutyl and tert-butyl.

Examples of R₁ and R₂ include hydrogen, methyl, ethyl, n-propyl, n-butyl, tert-butyl, hydroxy, methoxy, ethoxy, n-propyloxy, n-butyloxy, benzyloxy, mercapto, methylthio, ethylthio, carboxy, methoxycarbonyl, ethoxycarbonyl, butoxycarbonyl, formyl, acetyl, ethylcarbonyl, guanidino, N-methylguanidino, N-n-butylguanidino, acetoxy, propionyloxy, butyryloxy, acetamino, propionyl amino, butyrylamino, amino, dimethylamino, dibutylamino, aminomethyl, benzyloxycarbonylaminomethyl, sulfamyl, dimethylsulfamyl, nitro, cyano, fluorine, chlorine, bromine, iodine, trifluoromethyl, methylenedioxy, phenylamino, 3,4-dimethylphenylamino, and 3-trifluoromethylphenylamino.

In preferred embodiments Z represents a covalent bond.

Preferably R₁ or R₂ represent hydrogen or a straight or branched alkyl group having from 1 to 4 carbon atoms.

In particularly preferred embodiments R₁ is hydrogen and R₂ is

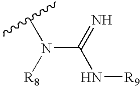

wherein R₈ and R₉ are hydrogen.

Preferably, the compound will be in the form of a pharmaceutically acceptable salt or ester.

The medicament may optionally further comprise one or more pharmaceutically acceptable carriers, excipients or diluents.

In certain embodiments, the medicament is in aqueous solution.

In certain embodiments of the medicament in aqueous solution, the medicament has a low concentration of chloride. Preferably, the medicament has a chloride concentration of less than 10 mM.

Preferably, the medicament in aqueous solution of low chloride concentration further comprises a pharmaceutically acceptable impermeant, monovalent anion in the range of from 100 mM to 160 mM, preferably in the range of from 140 mM to 150 mM. Preferably the pharmaceutically acceptable impermeant, monovalent anion is gluconate.

Most preferably, the compound is 6-amidino-2-napthyl 4-guanidinobenzoate dihydrochloride or a pharmaceutically acceptable salt or ester thereof. Examples include but are not limited to 6-amidino-2-napthyl 4-guanidinobenzoate dihydrochloride and 6-amidino-2-napthyl 4-guanidinobenzoate mesylate.

In preferred embodiments the amidino compound according to formula I is the sole active ingredient.

The intended respiratory diseases include diseases of the lung, airways (bronchioles, bronchi), and upper respiratory tract (nose, para-nasal sinuses, Eustachian tube and middle ear) but are not limited to those diseases that are characterised by poor mucociliary clearance and/or mucostasis.

Preferably the disease is selected from the group comprising cystic fibrosis (CF); bronchiectasis; chronic bronchitis; chronic obstructive pulmonary disease (COPD); rhino-sinusitis and otitis media.

Preferably, the respiratory disease is cystic fibrosis.

Some embodiments of the medicaments of the invention include other active agents selected from the group of antibiotics, vaccines, decongestants (nasal or bronchial), mucolytic agents, rhDNase, non-steroidal antiinflamatory agents (NSAIDs), steroids, antiviral agents, elastase inhibitors, exofacial sodium channel blocking agents, gene therapy agents, chloride channel activators, mannitol, purinergic receptor agonists and bronchodilators.

In one embodiment of the invention, the medicament is formulated for administration to the respiratory system by the pulmonary route.

Preferably, the medicament is formulated for administration by a method including but not limited to intratracheal instillation (delivery of solution into the airways by syringe), intratracheal delivery of liposomes, insufflation (administration of powder formulation by syringe or any other similar device into the airways), nebulization, dry powder inhalation and aerosol inhalation.

Aerosols (e.g. , jet or ultrasonic nebulizers, metered-dose inhalers (MDIs), and dry-powder inhalers (DPIs) can also be used in intranasal applications as well as for pulmonary administration.

Aerosol formulations are stable dispersions or suspensions of solid material and liquid droplets in a gaseous medium and can be placed into pressurized acceptable propellants, such as hydrofluoroalkanes (HFAs, i.e. HFA-134a and HFA-227, or a mixture thereof), dichlorodifluoromethane (or other chlorofluorocarbon propellants such as a mixture of Propellants 11,12, and/or 114), propane, nitrogen, and the like.

Preferably the medicament is formulated for administration by dry powder inhalation or aerosol inhalation together with a propellant selected from the group comprising hydrofluoroalkanes, chlorofluorocarbons, propane, nitrogen, or a mixture thereof.

In preferred embodiments the medicaments of the invention are formulated for the treatment of the nasal epithelium, para-nasal sinuses, the Eustachian tube and middle ear, and are formulated as solutions for delivery as drops, by pipette or by syringe to the nasal epithelium, para-nasal sinuses, the Eustachian tube and middle ear.

In further embodiments for the treatment of rhino-sinusitis, the medicament is formulated as a solution for the irrigation of the para-nasal sinuses by local instillation of said solution via cannula tube or syringe needle.

In another embodiment for the treatment of otitis media, the medicament is formulated as a solution for delivery as solution directly applied to the middle ear via the external auditory meatus and canal.

In a further aspect, the current invention relates to a method of treating respiratory disease, comprising administration of a therapeutically effective amount of an amidino compound formula I to a patient in need thereof.

Preferably, the compound will be in the form of a pharmaceutically acceptable salt or ester.

More preferably, the compound is administered together with one or more pharmaceutically acceptable carriers or diluents.

In certain embodiments, the compound is administered as an aqueous solution.

In certain embodiments of the compound in aqueous solution, the aqueous solution has a low concentration of chloride. Preferably, the total chloride concentration administered is less than 10 mM.

Preferably, the amidino compound in aqueous solution of low chloride concentration is administered together with a pharmaceutically acceptable impermeant, monovalent anion with a concentration in the range of from 100 mM to 160 mM, preferably in the range of from 140 mM to 150 mM. Preferably, the pharmaceutically acceptable impermeant, monovalent anion is gluconate.

Most preferably, the compound is 6-amidino-2-napthyl 4-guanidinobenzoate dihydrochloride or a pharmaceutically acceptable salt or ester thereof. Examples include but are not limited to 6-amidino-2-napthyl 4-guanidinobenzoate dihydrochloride and 6-amidino-2-napthyl 4-guanidinobenzoate mesylate.

Preferably, the respiratory disease to be treated is characterised by poor mucociliary clearance and/or mucostasis.

Preferably, the disease is selected from the group comprising cystic fibrosis (CF); bronchiectasis; chronic bronchitis; chronic obstructive pulmonary disease (COPD); rhino-sinusitis and otitis media. Most preferably, the respiratory disease is cystic fibrosis.

Preferably the administration is to the respiratory system by the pulmonary route.

Such an administration may be achieved by a method including but not limited to intratracheal instillation (delivery of solution into the airways by syringe), intratracheal delivery of liposomes, insufflation (administration of powder formulation by syringe or any other similar device into the airways), nebulization, dry powder inhalation and aerosol inhalation. Most preferably, the administration is by dry powder inhalation or aerosol inhalation together with a propellant selected from the group comprising hydrofluroalkanes, chlorofluocarbons, propane, nitrogen, or a mixture thereof.

In a further embodiment, the amidino compounds of formula I may be used in the treatment of conditions benefiting from lowered airway epithelial sodium channel activity.

Alternatively, the amidino of formula I may be used in the treatment of conditions benefiting from increased ciliary transport in the airways of a patient.

In yet another embodiment, the invention relates to pharmaceutical compositions comprising amidino compounds of Formula I, formulated for delivery to the respiratory system.

Preferably, the amidino compounds comprised in the pharmaceutical compositions of the invention are in the form of a pharmaceutically acceptable salt or ester.

More preferably, the pharmaceutical compositions of the invention also comprise one or more pharmaceutically acceptable carriers or diluents.

In certain embodiments, the pharmaceutical composition is in the form of an aqueous solution.

In certain embodiments of the pharmaceutical composition in aqueous solution, the pharmaceutical composition has a low concentration of chloride. Preferably, the pharmaceutical composition has a chloride concentration of less than 10 mM.

Preferably, the pharmaceutical composition in aqueous solution of low chloride concentration further comprises a pharmaceutically acceptable impermeant, monovalent anion in the range of from 100 mM to 160 mM, preferably in the range of from 140 mM to 150 mM. Preferably, the pharmaceutically acceptable impermeant, monovalent anion is gluconate.

Most preferably, the amidino compound comprised in the pharmaceutical compositions of the invention is 6-amidino-2-napthyl 4-guanidinobenzoate or a pharmaceutically acceptable salt or ester thereof.

The pharmaceutical compositions of the invention may be formulated for administration to the respiratory system by the pulmonary route.

Preferably, the pharmaceutical compositions of the invention are formulated for administration by any one of intratracheal instillation, intratracheal delivery of liposomes, insufflation, nebulization, dry powder inhalation and aerosol inhalation.

Most preferably, the pharmaceutical compositions of the invention are formulated for administration by dry powder inhalation or aerosol inhalation and further comprise a propellant selected from the group comprising hydrofluroalkanes, chlorofluocarbons, propane, nitrogen, or a mixture thereof

The current invention further relates to an inhalation device loaded with a pharmaceutical composition of the current invention. Preferably, the inhalation device is a dry powder inhaler, metered dose inhaler, jet nebulizer or ultrasonic nebulizer.

### DESCRIPTION OF THE FIGURES

The invention will be now be described by way of exemplification only and with reference to the accompanying figures, brief descriptions of which follow:

FIG. 1:

Short circuit current measurement of human bronchial epithelial (HBE) cells 24 d in culture, 21 d air-liquid interface (ALI). No pre-treatment prior to mounting in Ussing chamber. At 29 min following assembly of the chamber the basolateral and apical sides were flushed through each with 30 ml of fresh Krebs-Ringer buffer whilst maintaining the volume on each side of the chamber at 5 ml buffer. At 24.5 min 5 μl of 10⁻² M-amiloride (final concentration 10 μM) was added to the apical chamber. At 36 min were flushed as previously with 30 ml of fresh Krebs-Ringer buffer.

ΔI_{sc} (amiloride addition)=9.2 μAmp

R_{T}=1092±64 Ohm/cm²

FIG. 2:

Short circuit current recordings of HBE cells 24 d (A&B) & 25 d (C&D) in culture, 19 & 20 d ALI. A & C: Controls. B & D: Pre-treated with 10⁻⁶ M 6-amidino-2-napthyl 4-guanidinobenzoate for 90 min prior to assembly in Ussing chambers and measurement of I_{sc}. Amiloride to a final concentration of 10 μM was added to the apical surface of cells at 30 min after washing of cells with Krebs-Ringer buffer and assembly in Ussing chambers (approximately 10 min from start of recording). Forskolin to a final concentration of 10 μM was added to both the apical and basolateral surface 7 min later.

FIG. 3:

A: Amiloride ΔI_{sc}, and B: Forskolin ΔI_{sc}, from short circuit current recordings from HBE cells cultured for 24 & 25 d, 19 & 20 d at ALI. Results are studies for two culture batches. Closed circles: Cultures treated and incubated with 1 μM 6-amidino-2-napthyl 4-guanidinobenzoate for 90 min. prior to wash with Krebs-Ringer buffer and assembly into Ussing chambers. Open circles: Non-treatment controls. Amiloride 10 μM final concentration was added to the apical surface 30 min. after Krebs-Ringer buffer wash and assembly of Ussing chambers. Forskolin was added apically and basolaterally at 37 min.

FIG. 4:

Dose-response study: Short circuit current response to 90 min treatment and incubation with 6-amidino-2-napthyl 4-guanidinobenzoate over dose range 3.10⁻⁶ M to 3.10⁻⁸ M (closed bars) for HBE cells cultured for 18 & 19 d, 15 & 16 d at ALI. Non-treatment controls (open bars). Amiloride 10 μM final concentration was added to the apical surface 30 min. after Krebs-Ringer buffer wash and assembly in Ussing chambers. Forskolin was added apically and basolaterally at 37 min. A: Amiloride ΔI_{sc} (mean±SEM), and B: Forskolin ΔI_{sc} (mean±SEM) from short circuit current recordings. Controls and treatment groups analysed by one-way ANOVA. Tukey's multiple comparison test used to further analyse amiloride ΔI_{sc} results.

FIG. 5:

Time of onset study: Amiloride ΔI_{sc} response to treatment and incubation with 10⁻⁶ M 6-amidino-2-napthyl 4-guanidinobenzoate over time range 5 min. to 60 min. (closed circles) for HBE cells cultured for 22 d, 19 d at ALI. Non-treatment controls (open circles). Amiloride 10 μM final concentration was added to the apical surface 30 min. after Krebs-Ringer buffer wash and assembly in Ussing chambers. Controls and treatment groups analysed by one-way ANOVA. Tukey's multiple comparison test used to compare groups.

FIG. 6:

Duration of amiloride ΔI_{sc} response: Amiloride ΔI_{sc} response of HBE cells cultured for 26 d, 23 d at ALI, treated with 10⁻⁶ M 6-amidino-2-napthyl 4-guanidinobenzoate for 60 min. (closed circles), followed by periods of washout with fresh culture medium: 0 min, 1 h15 min, 3 h00 min and 4 h30 min. Non-treatment controls (open circles). Amiloride 10 μM final concentration was added to the apical surface 30 min. after Krebs-Ringer buffer wash and assembly in Ussing chambers. Controls and treatment groups analysed by one-way ANOVA. Tukey's multiple comparison test used to compare groups.

FIG. 7:

Short circuit current recordings of HBE cells 26 & 27 d in culture, 23 & 24 d ALI. A: Controls. B to E: Treated with 10⁻⁶ M 6-amidino-2-napthyl 4-guanidinobenzoate for 60 min, followed by a period of washout with fresh culture medium for—B: 0 min. C: 1 h 15 min. D: 3 h 00 min. E: 4 h 30 min. (Amiloride ΔI_{sc}data extracted for preparation of FIG. 6). Amiloride 10 μM final concentration was added to the apical surface at 30 min. after Krebs-Ringer buffer wash and assembling in Ussing chambers, forskolin was added apically and basolaterally at 37 min.

FIG. 8:

Amiloride ΔI_{sc} response of HBE cells treated with 6-amidino-2-napthyl 4-guanidinobenzoate metabolites. A: HBE cells cultured for 22 d, 17 d at ALI. Treated for 90 min with 10⁻⁶ M- or 10⁻⁴ M-pGBA (closed triangles), or 10⁻⁶ M 6-amidino-2-napthyl 4-guanidinobenzoate (closed circles). Non-treatment controls (open circles). B: HBE cells cultured for 34 & 35 d, 28 & 29 d at ALI. Treated for 90 min with 10⁻⁴ M- or 10⁻⁶ M-AN (closed inverted triangles), mixture of 10⁻⁶ M-metabolites pGBA & AN (closed diamonds) or 10⁻⁶ M 6-amidino-2-napthyl 4-guanidinobenzoate (closed circles). Non-treatment controls (open circles). Amiloride 10 μM final concentration was added to the apical surface 30 min. after Krebs-Ringer buffer wash and assembly in Ussing chambers. Controls and treatment groups analysed by one-way ANOVA, t-test used to compare control and 6-amidino-2-napthyl 4-guanidinobenzoate treated groups.

FIG. 9:

Nasal PD responses to perfusion with 10⁻⁵ M-amiloride. Responses were measured in two periods before perfusion with 3.10⁻⁶ M-6-amidino-2-napthyl 4-guanidinobenzoate; and in three periods after perfusion with 3.10⁻⁶ M-6-amidino-2-napthyl 4-guanidinobenzoate.

FIG. 10:

Incidence of mucus host-defence episodes during the perfusion schedule for study of nasal PD in each of four experiments. Responses were scored 1 to 3 based on the combination of symptoms and the magnitude of the response for each episode.

### DETAILED DESCRIPTION OF INVENTION

The current inventors have found 6-amidino-2-napthyl 4-guanidinobenzoate to be a potent long duration inhibitor of ENaC activity in airway epithelial cells. This and related compounds therefore have potential in the treatment of respiratory diseases characterised by mucostasis such as cystic fibrosis (CF); bronchiectasis; chronic bronchitis; chronic obstructive pulmonary disease (COPD); rhino-sinusitis and otitis media.

The preferred respiratory disease for treatment with such compounds is cystic fibrosis.

### Potency:

The profile of activity of 6-amidino-2-napthyl 4-guanidinobenzoate shows similarities to the profiles reported for the Kunitz-domain serine protease inhibitors BAY 39-9437 and aprotinin (Bridges et al. 2001). The current inventors have achieved a maximum down regulation of ENaC activity with 6-amidino-2-napthyl 4-guanidinobenzoate in the range 50% to 70% of the inhibition achieved by application of high dose amiloride to human airway epithelial cell cultures. BAY 39-9437 and aprotinin achieved efficacy in the same range (Bridges et al. 2001) compared with high dose amiloride. The half maximal inhibitory concentration for 6-amidino-2-napthyl 4-guanidinobenzoate is in the range 30 to 100 nM, which compares favourably to BAY 39-9437 (reported as approximately 25 nM).

Unlike with amiloride, onset of down regulation of ENaC activity by serine protease inhibitors is not immediate. 6-amidino-2-napthyl 4-guanidinobenzoate achieves half maximal inhibition by 5 min and maximum down regulation by 30 min. Again, this compares favourably with BAY 39-9437 where an onset t_{1/2} of 45 min was reported (Bridges et al 2001).

### Duration of Action:

An important aspect to the profile of 6-amidino-2-napthyl 4-guanidinobenzoate is its long duration of action. Significant down regulation of ENaC is still observed 1 h 45 min after washout of the 6-amidino-2-napthyl 4-guanidinobenzoate, which represents 78% of the maximal down regulation observed at 30 min after washout.

Cells treated with 6-amidino-2-napthyl 4-guanidinobenzoate and transferred to fresh culture for 3 h 00 min prior to assembly into Ussing chambers for short circuit current recording show, in the first 15 minutes of recording, a slope of rising baseline short circuit current that is over 20 times that recorded for untreated controls (FIGS. 7D and 7A). The increased slope of the rising baselines in these cells suggests that activation of ENaC is triggered at the start of recording by the actions of assembling the cell cultures into the Ussing chambers and initiating voltage clamping. This interpretation of the recordings suggests that the duration of down regulation of ENaC after washout of 6-amidino-2-napthyl 4-guanidinobenzoate is considerably underestimated for undisturbed cell cultures.

The long duration of down regulation achieved by 6-amidino-2-napthyl 4-guanidinobenzoate again compares favourably with BAY 39-9437 where a half-life of 15 min. was reported (Bridges et al. 2001).

The pharmacological profile of 6-amidino-2-napthyl 4-guanidinobenzoate as a potent long duration down regulator of epithelial sodium channel activity in human airway epithelial cells is not predicted by previous studies.

The term “respiratory diseases” includes but is not limited to conditions of the lung, airways (bronchioles, bronchi), and upper respiratory tract (nose, para-nasal sinuses, Eustachian tube and middle ear) characterised by impaired mucociliary clearance, possibly resulting in mucostasis. Such conditions include but are not limited to: cystic fibrosis (CF); bronchiectasis; chronic bronchitis; chronic obstructive pulmonary disease (COPD); rhino-sinusitis; otitis media.

The term “impermeant” refers to solutes that are unable to pass through a cell membrane.

The current invention will be further understood by way of the following examples.

### General Methods

Examples 1 to 6 use the same set of material and methods, which are described below.

### Cells

Human bronchial epithelial (HBE) cells were purchased from Cambrex Bioscience Ltd, UK., and were stored frozen in liquid nitrogen until required for use.

### Cell Culture

HBE cells were amplified by growing in secondary culture according to the suppliers' instructions. Cells were seeded at densities of between 3,000 to 4,000/cm² in plastic T-25 culture flasks in bronchial epithelial cell growth medium (BEGM) prepared from bronchial epithelial basal medium (BEBM, Cambrex, UK) supplemented with bovine pituitary extract, hydrocortisone, human epidermal growth factor, epinephrine, transferrin, insulin, retinoic acid, triiodothyronine, gentamicin, and amphotericin B. Cultures were maintained at 37° C. in 5% CO₂ in a humidified incubator. Medium was changed every 1 to 3 days until cells were approximately 90% confluent. Cells were then passaged with trypsin EDTA according to the suppliers' instructions, and either used for further cell cultures, or cryopreserved in liquid nitrogen in BEGM containing 10% foetal bovine serum and 10% DMSO.

HBE cells were prepared for electrophysiology studies by growing as tertiary cultures on Snapwell (Corning) 0.4μ clear polyester membrane inserts 1.2 cm² coated with collagen IV (Sigma). Cells were seeded at approximately 10⁵/cm² in 0.5 ml 1:1 DMEM (Cambrex, UK) BEGM containing bovine pituitary extract (50 μg/ml), hydrocortisone (0.5 μg/ml), human epidermal growth factor (0.5 ng/ml), epinephrine (0.5 μg/ml), transferrin (10 μg/ml), insulin (5 μg/ml), retinoic acid (50 nM), and gentamicin (50 μg/ml) (Danahay medium; Danahay et al. 2002). Snapwell inserts were mounted in 6-well plates with 3 ml Danahay medium in the wells, and grown at 37° C. in 5% CO₂ in a humidified incubator. Medium was changed every 1 to 3 days. An apical air-liquid interface (ALI) was established as the cells achieved confluence (3 to 6 days) by removal of medium from the centre of the insert. Confluent differentiated bronchial epithelial cells were used for electrophysiology studies at between 15 and 29 days after establishing ALI.

### Drug Treatment Protocols and Measurement of Short Circuit Current (I_{sc})

Differentiated HBE cells grown to confluence at an air-liquid interface for 15 to 29 days were used to study substances for ability to achieve long duration down regulation of the epithelial amiloride-sensitive sodium channel (ENaC).

Step 1: Treatment with substance. For each group of cells treated with a substance, 30 μl of 100 times final concentration of substance was added to the 3 ml basolateral culture medium, mixed and 150 μl of the resultant basolateral fluid was transferred to the apical surface for 5 min. For non-treatment control groups of basolateral fluid with no added substance was transferred to the apical surface. After 5 min. equilibration the excess apical fluid was then transferred back to the basolateral side of the culture well.

Step 2: Variable period of incubation with the substance. Dependent on the individual experiment the cells were returned to the 37° C. incubator for periods of 0 min. to 120 min.

Step 3: Period of substance washout with fresh culture medium. For experiments to study the duration of a substance response the HBE cells on Snapwell inserts were washed with fresh culture medium (no added substance) at 37° C. and placed in fresh 6-well plates containing 3 ml fresh culture medium (no added substance) on the basolateral side. Cells were returned to the 37° C. incubator for periods of 0 minutes to 120 minutes. Step 3 was omitted in studies where the duration of the substance response was not investigated.

Step 4: Wash with Krebs-Ringer buffer and assembly in Ussing chambers. The Snapwell insert cultures of HBE cells were removed from the 6-well plates and washed with excess Krebs-Ringer buffer (120 mM-NaCl, 25 mM-NaHCO₃, 3.3 mM-KH₂PO₄, 0.8 mM-K₂HPO₄, 1.2 mM-CaCl₂, 1.2 mM-MgCl₂, 10 mM-glucose) pH7.4 at 37° C. The inserts were then mounted in Ussing chambers (Snapwell diffusion chambers, Costar UK Ltd.). Each side of the chamber was filled with 5 ml Krebs-Ringer buffer and gassed continuously with 95% O₂/5% CO₂ at 37° C.

Step 5: Short circuit current (I_{sc}) measurement. Cells were voltage clamped to 0 mV using a WPI EVC 4000 voltage clamp (World Precision Instruments, UK). Silver/silver chloride electrodes (Costar Ltd., UK) were used to monitor I_{sc}, which was recorded continuously throughout each experiment using AcqKnowledge III for MP100WSW data acquisition software (Biopac, Linton Instruments, UK). Transepithelial resistance (R_{T}) was determined every minute by delivering a 5 mV pulse for 3 seconds and recording the change in I_{SS}, over the 3 second period (ΔI_{sc}). R_{T} was was calculated using the Ohm's law relationship R_{T}=[V(5 mV)/ΔI_{sc}].

At 30 min. after assembling the cultured HBE cells in Ussing chambers, 5 μl of 10⁻² M-amiloride (final chamber concentration 10 μM) was added to the apical surface. ENaC short circuit current activity was determined for each culture by measuring the difference between I_{sc} immediately prior to addition of amiloride and at 3 min. following addition of the agent (Amiloride ΔI_{sc}).

At 37 min. 5 μl of 10⁻² M-forskolin (final chamber concentration 10 μM) was added to both the apical and basolateral surfaces. Forskolin stimulated a cAMP-dependent chloride channel current by activation of the cystic fibrosis transmembrane regulator (CFTR) in the epithelial cells. The magnitude of the CFTR I_{sc} response to forskolin was determined for each culture by measuring the difference between I_{sc} immediately prior to addition of forskolin and at plateau at approximately 6 min. following addition of the agent (Forskolin ΔI_{sc})

### Data Analysis

An unpaired t-test was used to analyse experiments where two data groups were compared; statistical significant difference level p≦0.05.

One-way analysis of variance (ANOVA) was used to analyse studies where more than two data groups were compared. When statistical significance was achieved p≦0.05 in ANOVA, Tukey's multiple comparison test was used to compare between individual groups.

### EXAMPLE 1

### Short Duration Inhibition of ENaC Activity by Amiloride

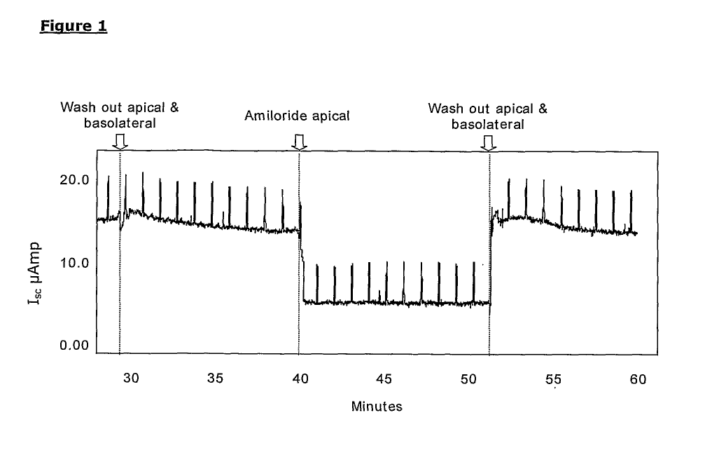

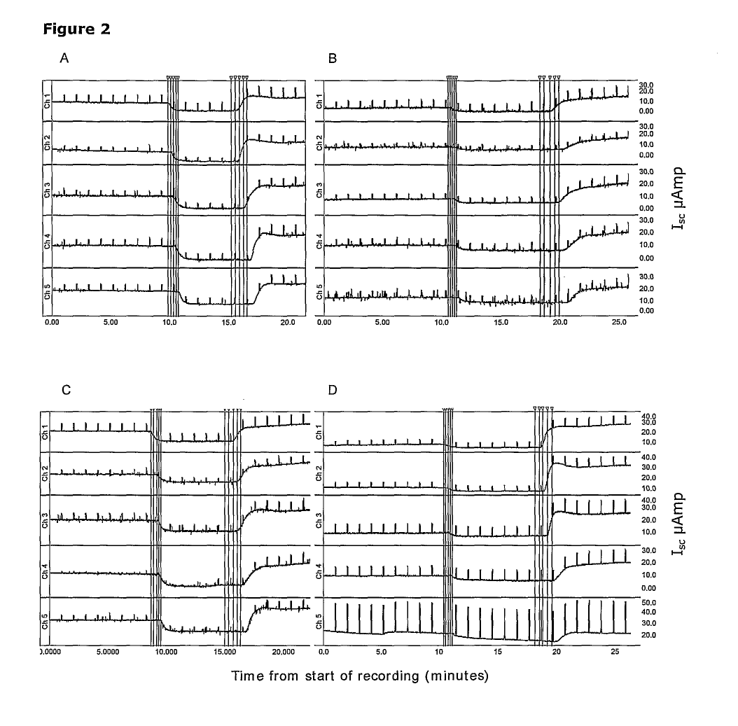

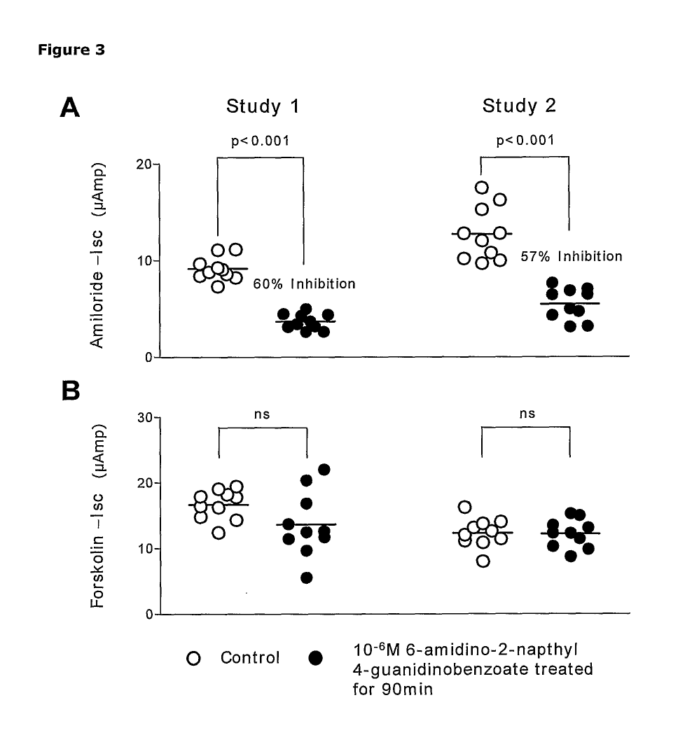

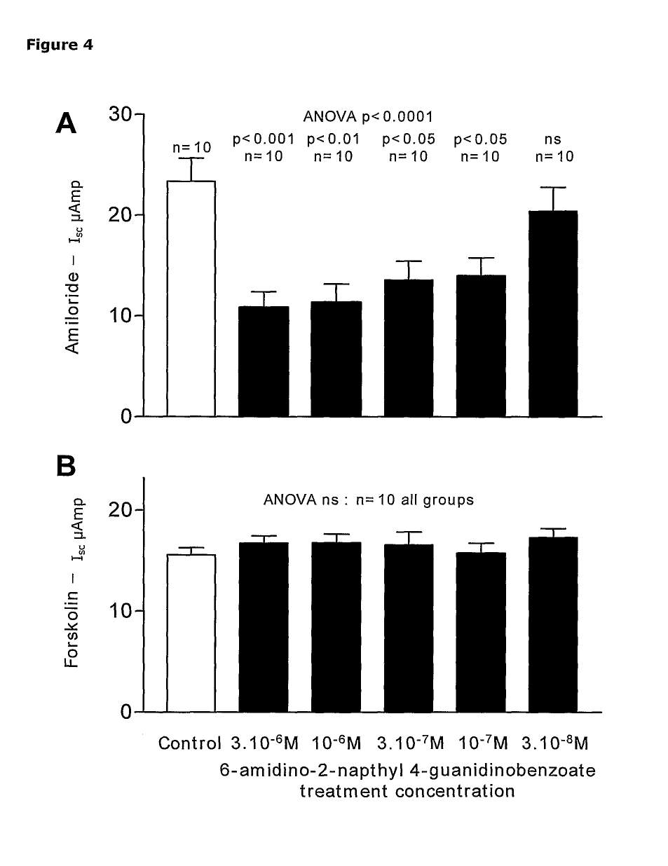

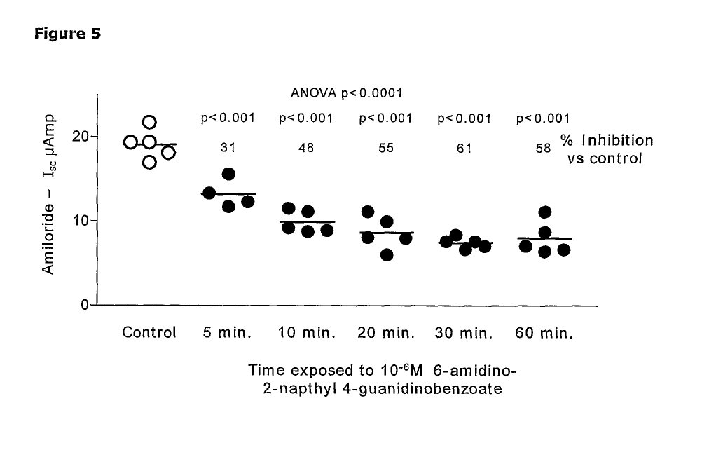

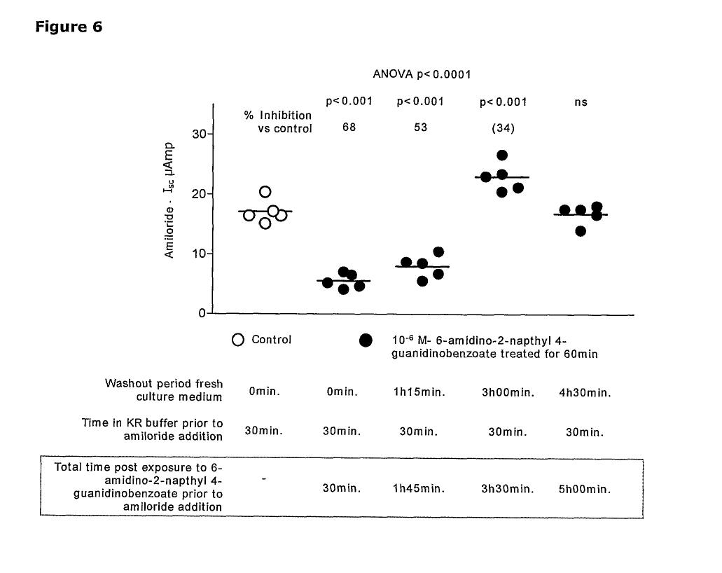

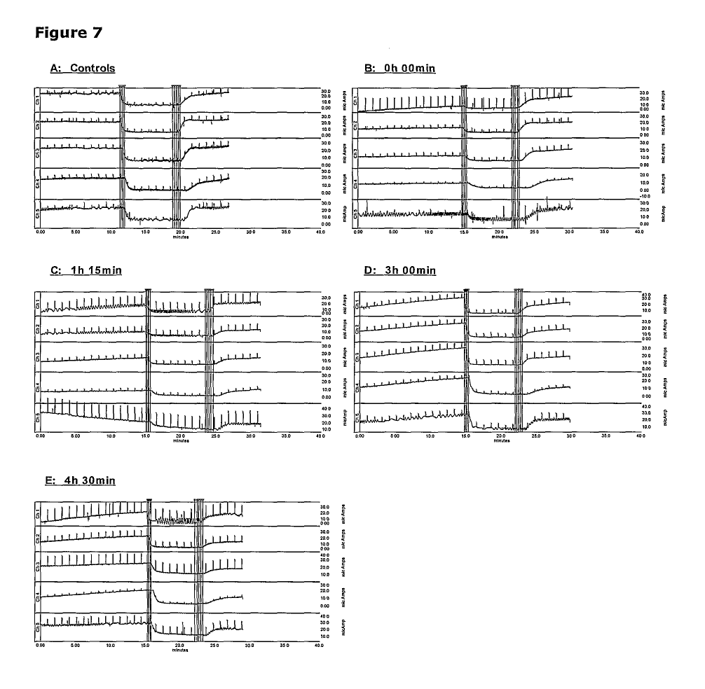

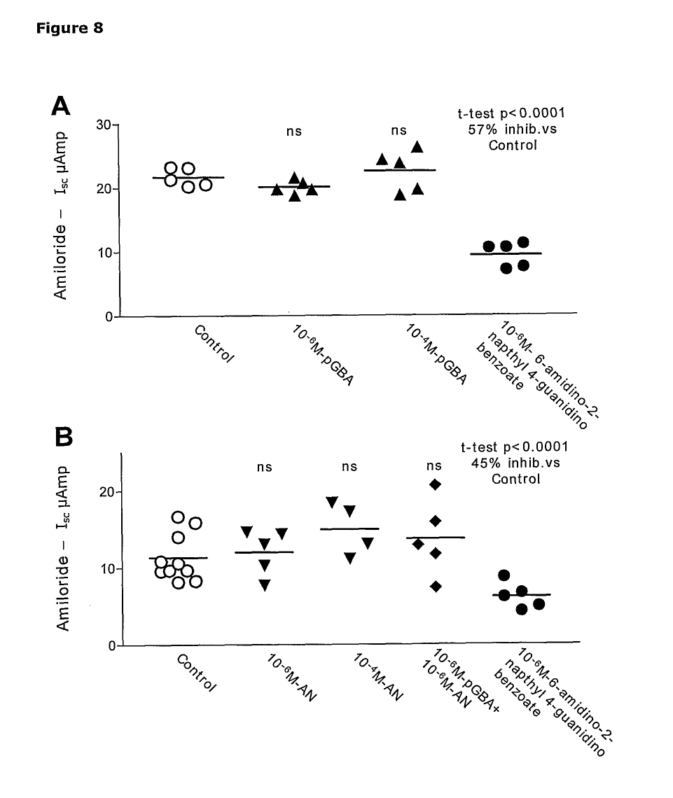

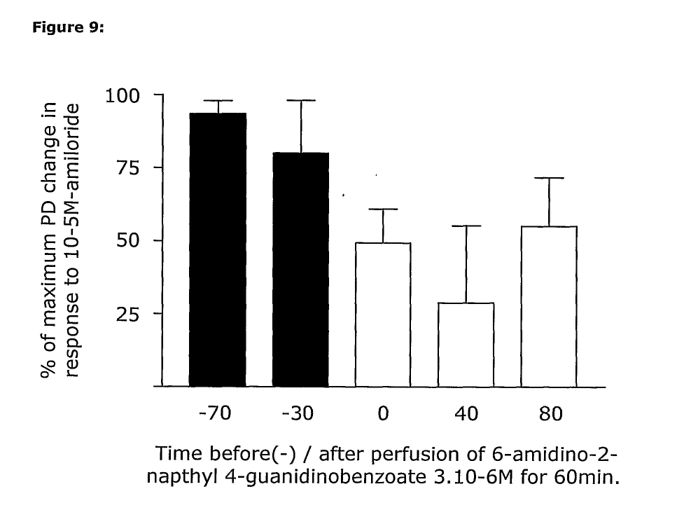

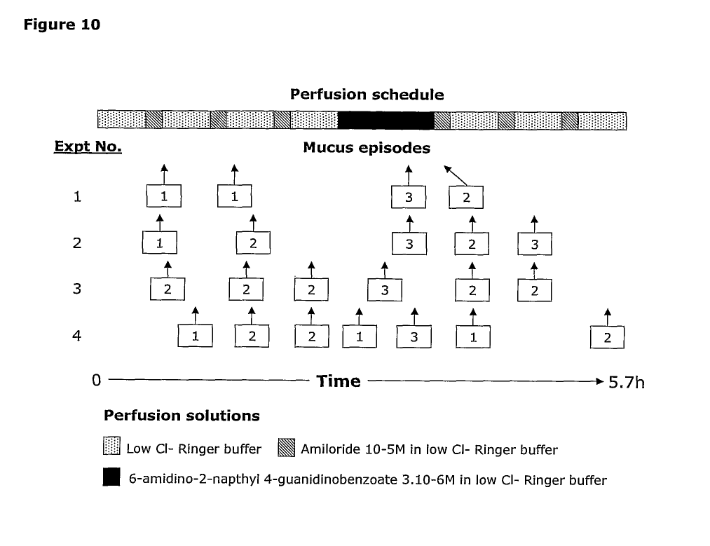

FIG. 1 illustrates the short circuit current response of HBE cells to 10 μM-amiloride added to the apical side of cells mounted in an Ussing chamber. Inhibition of ENaC activity occurred rapidly. Typically short circuit current was reduced to a new plateau level within 2 min of application of amiloride to the apical surface.

When amiloride was removed from contact with the cell preparation by washout with fresh Krebs-Ringer buffer of both the apical and basolateral sides of the Ussing chamber, short circuit current increased rapidly to plateau within 2 to 5 min. (FIG. 1) close to the pre-amiloride current level.

### EXAMPLE 2

### Pre-Treatment of HBE Cells for 90 min with 1 μM 6-amidino-2-napthyl 4-guanidinobenzoate

HBE cells cultured for 24 & 25 days, and 19 & 20 days at ALI were pre-treated for 90 min with 1 μM 6-amidino-2-napthyl 4-guanidinobenzoate in culture medium. Control cultures were treated according to the same protocol, but without 6-amidino-2-napthyl 4-guanidinobenzoate in the culture medium. After 90 min the substance treated cultures were washed free of 6-amidino-2-napthyl 4-guanidinobenzoate with Krebs-Ringer buffer, and then assembled in Ussing chambers with 5 ml buffer each side of the chamber. Again control cultures were treated according to the same protocol.

At 30 min following assembly of the Ussing chambers 10 μM-amiloride was added to the apical surface of cells. The resulting fall in short circuit current (amiloride ΔI_{sc}) in response to application of amiloride (approximately 10 min. after start of recording) was greater in control cultures (FIGS. 2A & 2C) compared to cultures pre-treated with 6-amidino-2-napthyl 4-guanidinobenzoate (FIGS. 2B & 2D). FIG. 3 illustrates two studies in which amiloride ΔI_{sc} was significantly (p<0.001) reduced, by 60% and 57%, in 1 μM 6-amidino-2-napthyl 4-guanidinobenzoate, 90 min pre-treated cultures (FIG. 3A) compared to control cultures. The reduction in amiloride ΔI_{sc} indicates reduced ENaC activity as the result of 6-amidino-2-napthyl 4-guanidinobenzoate treatment. It should also be noted that the reduced ENaC activity was seen 30 min after 6-amidino-2-napthyl 4-guanidinobenzoate had been washed away from the cultured cells. This demonstrates that 6-amidino-2-napthyl 4-guanidinobenzoate exerts a long duration down regulatory effect on the epithelial sodium channel in HBE cells.

Amiloride was not washed away from the cultures after plateau (FIG. 2), and at 37 min following assembly of the Ussing chambers 10 μM-forskolin was added to both the apical and basolateral sides of the chambers. Short circuit current was observed to increase (forskolin ΔI_{sc}) in all cultures (FIG. 2). The increase in short circuit current in response to forskolin is consistent with activation of CFTR anion conductance in HBE cells. FIG. 3 illustrates that in contrast to the amiloride ΔI_{sc}, 1 μM 6-amidino-2-napthyl 4-guanidinobenzoate, 90 min pre-treatment had no significant effect on forskolin ΔI_{sc} compared to control (FIG. 3B).

### EXAMPLE 3

### Dose-Response to 6-amidino-2-napthyl 4-guanidinobenzoate Treatment

HBE cells cultured for 18 & 19 days, and 15 & 16 days at ALI were pre-treated for 90 min with 6-amidino-2-napthyl 4-guanidinobenzoate over the dose range 30 nM to 3 μM in culture medium. Amiloride ΔI_{sc} for 6-amidino-2-napthyl 4-guanidinobenzoate treated cultures was reduced compared to control in a dose related manner (FIG. 4A). Pre-treatment with 3 μM-6-amidino-2-napthyl 4-guanidinobenzoate resulted in 54% reduction in amiloride ΔI_{sc} (p<0.001); 1 μM in 52% reduction (p<0.01); 0.3 μM in 42% reduction (p<0.05); 0.1 μM in 40% reduction (p<0.05); and 30 nM in 13% reduction (ns) (FIG. 4A). Again the reduced amiloride ΔI_{sc} response was seen 30 min after 6-amidino-2-napthyl 4-guanidinobenzoate had been washed away from the cultured cells.

FIG. 4B illustrates that 6-amidino-2-napthyl 4-guanidinobenzoate pre-treatment (90 min) across the dose range 30 nM to 3 μM was without effect on forskolin ΔI_{sc} compared to control.

### EXAMPLE 4

### Time of Onset Study of Down Regulation of Amiloride ΔI_{sc}

HBE cells cultured for 22 days, 19 days at ALI, were studied to assess down regulation of amiloride ΔI_{sc} in response to exposure to 1 μM 6-amidino-2-napthyl 4-guanidinobenzoate in culture medium for pre-treatment times over the range 5 min to 60 min. Again after pre-treatment the cultures were washed free of 6-amidino-2-napthyl 4-guanidinobenzoate with Krebs-Ringer buffer before mounting in Ussing chambers. FIG. 5 shows the time dependent relationship in the amiloride ΔI_{sc} response. Significantly reduced amiloride ΔI_{sc} response was observed at all time points studied; 31% reduction vs control (p<0.001) at 5 min pre-treatment, 48% (p<0.001) at 10 min, 55% (p<0.001) at 20 min, 61% (p<0.001) at 30 min, and 58% (p<0.001) at 60 min.

No significant difference between treatment and control was observed for the forskolin AI, response over the pre-treatment time range studied.

### EXAMPLE 5

### Duration of the Amiloride ΔI_{sc} Response

The effects of time were further studied by evaluating the effect of a period of washout on amiloride ΔI_{sc} responses. HBE cells cultured for 26 days, 23days at ALI, were pre-treated with 1 μM 6-amidino-2-napthyl 4-guanidinobenzoate for 60 min. Cells were then washed free of 6-amidino-2-napthyl 4-guanidinobenzoate with fresh culture medium and returned to the incubator for between 0 min and 4 h 30 min. Cells were then washed as before with Krebs-Ringer buffer before mounting in Ussing chambers.

FIG. 6 shows that culture medium washout periods of 0 min and 1 h 15 min resulted in significantly reduced amiloride ΔI_{sc} compared to control; 68% reduction vs control (p<0.001) for 0 min culture medium washout, 53% (p<0.001) for 1 h 15 min. It should again be noted that the 30 min. time interval between wash with Krebs-Ringer buffer and addition of amiloride means that the total time elapsed after removal of 6-amidino-2-napthyl 4-guanidinobenzoate was 30 min and 1 h 45 min respectively for these two groups.

By contrast the amiloride ΔI_{sc} for the 3 h 00 min culture medium washout period was elevated (+34%, p<0.001) compared to control (FIG. 6). However inspection of the short circuit current recordings (FIG. 7) illustrates a further complexity in interpretation of the duration of response data. In FIG. 7A it can be seen that the baseline I_{sc} for the five control cultures is relatively stable over the initial recording period 0 to 12 min prior to addition of amiloride, average baseline slope +0.04 μAmp/min. This contrasts particularly with the short circuit current recordings for the equivalent period in FIG. 7D; 6-amidino-2-napthyl 4-guanidinobenzoate pre-treated cultures followed by 3 h 00 min washout with culture medium. Here there is a rising baseline I_{sc} for the initial period in all five cultures, average baseline slope +0.93 μAmp/min., but this is followed by a stable plateau after inhibition of ENaC by addition of amiloride. This pattern suggests that transfer of the cells from culture medium to Krebs-Ringer buffer and voltage clamping the cultures at 0 mV may have triggered activation of sodium channels at this point in the 3 h washout cells.

FIG. 6 further shows that for the 4 h 30 min culture medium washout group amiloride ΔI_{sc} was not significantly different to control. However the rising baseline in the initial period of short circuit current recording (FIG. 7E), average baseline slope +0.60 μAmp/min., again suggests triggering of activation of sodium channels.

### EXAMPLE 6

### 6-amidino-2-napthyl 4-guanidinobenzoate Metabolites Do Not Down Regulate Amiloride ΔI_{sc}

6-amidino-2-napthyl 4-guanidinobenzoate is known to be metabolised in plasma to compounds p-guanidinobenzoic acid (pGBA) and 6-amidino-2-naphthol (AN). HBE cells cultured for 22 days, 17 days at ALI were pre-treated for 90 min with 1 μM or 100 μM-pGBA, or 1 μM 6-amidino-2-napthyl 4-guanidinobenzoate. Amiloride ΔI_{sc} for the pGBA treated groups was not significantly different from control (FIG. 8A). Cultures pre-treated with 1 μM 6-amidino-2-napthyl 4-guanidinobenzoate for 90 min showed amiloride ΔI_{sc} reduced by 57% (p<0.0001) compared to control.

HBE cells cultured for 34 & 35 days, 28 & 29 days at ALI were pre-treated for 90 min with 1 μM or 100μM-AN, or 1 μM-pGBA plus 1 μM-AN, or 1 μM 6-amidino-2-napthyl 4-guanidinobenzoate. Amiloride ΔI_{sc} for the AN treated groups and for the combined pGBA and AN group was not significantly different from control (FIG. 8B). Cultures pre-treated with 1 μM 6-amidino-2-napthyl 4-guanidinobenzoate for 90 min showed amiloride ΔI_{sc} reduced by 45% (p<0.0001) compared to control.

Various aspects of the present invention have been exemplified in the preceding part of the specification. These formulations and methods discussed are intended for exemplification purposes only and are not intended to limit the scope of the accompanying claims.

### EXAMPLE 7

### Perfusion of Human Nasal Epithelium with 6-amidino-2-napthyl 4-guanidinobenzoate

Preliminary experiments to monitor effects of perfusion of 6-amidino-2-napthyl 4-guanidinobenzoate on the nasal epithelium on the amiloride-sensitive (sodium-dependent) component of nasal PD in a volunteer normal human subject have been carried out. The following methods were used:

### Methods

The method of measurement of nasal PD was an adaptation of that described by Standaert et al. (2004).

### Solutions

Ringer/HEPES buffer solution was prepared containing 148 mM-NaCl, 4.05 mM-KCl, 1.2 mM-CaCl₂, 1.2 mM-MgCl₂, 2.4 mM-K₂HPO₄, 0.4 mM-KH₂PO₄, 10 mM-HEPES, and adjusted to pH 7.4. Low chloride Ringer/HEPES buffer solution was prepared by replacing NaCl with 148 mM-Na gluconate in the Ringer solution components.

### Electrodes and Equipment

Agar electrodes were prepared with 3.7% agarose dissolved in Ringer/HEPES buffer pH 7.4. The reference electrode was placed and taped in contact with a small area on the forearm where the epidermis had been removed by gentle abrasion. A dual lumen nasal recording electrode was prepared using a lm length of soft plastic tubing 2.4 mm diameter, into which 1 mm diameter polythene tubing was inserted through the wall and along the lumen for 15 cm from one end. The 2.4 mm diameter tubing was filled with 3.7% agarose dissolved in Ringer/HEPES buffer pH 7.4. The 1 mm diameter tubing was used for perfusion of buffer solutions and drugs to the nasal epithelium. The ends of the agar electrodes were each placed in contact with a calomel half-cell in 3 M-KCl solution. Voltages were measured by connecting the calomel electrodes to a high impedance voltmeter (Keithley 617 programmable electrometer). Output from the voltmeter was recorded by connection to a Biopac MP100 data aquisition unit and PC running Acqknowledge III data acquisition software.

### Nasal PD Measurement

Nasal PD was recorded via the recording electrode placed in contact with the nasal epithelium approximately 4 cm inside the nostril. The nasal epithelium was perfused according to the following schedule and nasal PD was recorded continuously.

1. Ringer/HEPES buffer was perfused at 0.5 ml/min for 5 min, and starting baseline PD was recorded.

2. Perfusion was then switched to low chloride Ringer/HEPES buffer pH 7.4 for 30 min during which time initial hyperpolarisation of the epithelium occurred.

3. The nasal epithelium was then perfused with 10⁻⁵ M-amiloride in low chloride Ringer/HEPES buffer pH 7.4 for 10 min. Measurement of the amiloride-sensitive (sodium-dependent) component of nasal PD was then made by measuring the difference between the average PD recorded over the 2 min before perfusion with amiloride and the average PD recorded in the period 4 to 6 min after perfusion with amiloride.

4. Amiloride was washed away from the nasal epithelium by perfusion with low chloride Ringer/HEPES buffer pH 7.4 for 30 min.

5. Steps 3 and 4 were then repeated twice more.

6. The nasal epithelium was then perfused with 3 μM 6-amidino-2-napthyl 4-guanidinobenzoate in low chloride Ringer/HEPES buffer pH 7.4 for 60 min.

7. Steps 3 and 4 were then repeated a further twice, followed by either a period of administration of amiloride (3 experiments), or a final repetition of both steps 3 and 4 (1 experiment).

### Results

### Nasal PD

The nasal PD protocol was performed on 4 separate days, a minimum of 2 days apart. In the initial period of perfusion with Ringer/HEPES buffer the mean nasal PD was −6.1 mV (range 0 to −10.4 mV). Perfusion with low chloride Ringer/HEPES buffer caused gradual hyperpolarization of the nasal epithelium. At the end of the second period of perfusion with low chloride Ringer/HEPES buffer the mean nasal PD was −13.1 mV (range −6.3 to −20.3 mV).

Over the 5 h time course of each experiment nasal PD drifted and oscillated. However, nasal PD was sufficiently stable over periods of 10 min to allow meaningful measurements of responses to perfusion of amiloride to be made. Characteristically perfusion of the nasal epithelium with 10⁻⁵ M-amiloride caused partial depolarisation of the epithelium that was observable within 2 min of initiation of perfusion of the drug, and plateaued within 4 to 6 min. Changes in nasal PD caused by perfusion of amiloride were calculated as the difference between the mean PD recorded in the 2 min period before perfusion with amiloride and the mean PD recorded in the plateau period 4 to 6 min after start of perfusion with amiloride. Within each experiment the change in nasal PD at each period of application of amiloride was expressed as a percentage of the maximum change observed in the experiment. FIG. 9 shows for 4 experiments the average percent of maximum PD change in response to 10⁻⁵ M-amiloride in the periods 1) 70 min before perfusion of 3.10⁻⁶ M-6-amidino-2-napthyl 4-guanidinobenzoate (94%); 2) 30 min before (80%); 3) immediately following perfusion of 3.10⁻⁶ M-6-amidino-2-napthyl 4-guanidinobenzoate for 60 min (49%); 4) 40 min after (29%); 5) 80 min after (55%).

### Mucus Host-Defence Responses

Perfusion of the nasal epithelium with low chloride Ringer/HEPES buffer imposes a chemical gradient for chloride secretion, and therefore also provides a drive for fluid secretion. Periods of perfusion with 10⁻⁵ M-amiloride and with 3.10⁻⁶ M-6-amidino-2-napthyl 4-guanidinobenzoate stimulated episodes of mucus host-defence responses characterised in any episode by recording one or more of the following features:
Post nasal sensation; drip and/or catarrh; taste
Mucus gel secretion exuding from nostril; perfused nostril and/or contralateral nostril
Sneeze(s)
Itch in nostril; unilateral or bilateral
Eye watering
Blockage of the nostril; unilateral or bilateral

Mucus host-defence episodes were scored 1 to 3 on the basis of the number of features and the magnitude of the responses observed. FIG. 10 reports the score of the episodes recorded, and indicates the time of their occurrence in the perfusion protocol.

### Conclusions

Perfusion of the nasal epithelium with 3.10⁻⁶ M-6-amidino-2-napthyl 4-guanidinobenzoate in low chloride Ringer/HEPES buffer pH 7.4 for 60 min reduced the amiloride-sensitive (sodium-dependent) component of nasal PD for up to 80 min following perfusion of 6-amidino-2-napthyl 4-guanidinobenzoate.
Periods of perfusion with either 10⁻⁵ M-amiloride or with 3.10⁻⁶ M-6-amidino-2-napthyl 4-guanidinobenzoate in low chloride Ringer/HEPES buffer stimulated episodes of mucus host-defence responses where a component of the response was consistent with ciliary clearance of mucus gel in the sinuses and on the nasal epithelium.
Observation of a mucus host-defence response to 6-amidino-2-napthyl 4-guanidinobenzoate is novel.
Highest scores for episodes of mucus host-defence response were recorded during periods of perfusion of 3.10⁻⁶ M-6-amidino-2-napthyl 4-guanidinobenzoate in low chloride Ringer/HEPES buffer.

### References

Bridges R J, Newton B B, Pilewski J M, Devor D C, Poll C T. & Hall R L.

Na+ transport in normal and CF human bronchial epithelial cells is inhibited by BAY 39-9437. Am J Physiol Lung Cell Mol Physiol. 2001; 281: L16-23.

Danahay H, Atherton H, Jones G, Bridges R J. & Poll C T.

Interleukin-13 induces a hypersecretory ion transport phenotype in human bronchial epithelial cells.

Am J Physiol Lung Cell Mol Physiol. 2002; 282: L226-236.

Hitomi Y, Ikari N, Fujii S

Inhibitory effect of a new synthetic protease inhibitor (FUT-175) on the coagulation system.

Haemostasis. 1985;15: 164-8

Iwaki M, Oda M, Ozeki M, Ino Y, Suzuki K, Koshiyama Y, Motoyoshi A, Ogihara M, Suzuki S, Fujita M, et al.

[Pharmacological studies of FUT-175, nafamstat mesilate. III. Anti-inflammatory activities of FUT-175].

Nippon Yakurigaku Zasshi. 1984;84: 373-84.

Iwashita K, Kitamura K, Narikiyo T, Adachi M, Shiraishi N, Miyoshi T, Nagano J, Tuyen DG, Nonoguchi H. & Tomita K.

Inhibition of prostasin secretion by serine protease inhibitors in the kidney.

J Am Soc Nephrol. 2003;14: 11-16.

Muto, S, Imai M. & Asano Y.

Effect of nafamostat mesilate on Na+ and K+ transport properties in the rabbit cortical collecting duct.

Br.J.Pharmacol. 1993; 109: 673-678.

Muto S, Imai M. & Asano Y.

Mechanisms of the hyperkalaemia caused by nafamostat mesilate: effects of its two metabolites on Na+ and K+ transport properties in the rabbit cortical collecting duct.

Br.J.Pharmacol. 1994; 111: 173-178.

Narikiyo T, Kitamura K, Adachi M, Miyoshi T, Iwashita K, Shiraishi N, Nonoguchi H, Chen L M, Chai K X, Chao J. & Tomita K.

Regulation of prostasin by aldosterone in the kidney.

J Clin Invest. 2002; 109: 401-408.

Rossier B C.

The epithelial sodium channel: activation by membrane-bound serine proteases.

Proc Am Thorac Soc. 2004; 1: 4-9.

Standaert T A, Boitano L, Emerson J, Milgram L J, Konstan M W, Hunter J, Berclaz P Y, Brass L, Zeitlin P L, Hammond K, Davies Z, Foy C, Noone PG, Knowles M R.

Standardized procedure for measurement of nasal potential difference: an outcome measure in multicenter cystic fibrosis clinical trials.

Pediatr Pulmonol. 2004;37(5):385-92.

Tong Z, Illek B, Bhagwandin V J, Verghese G M. & Caughey G H.

Prostasin, a membrane-anchored serine peptidase, regulates sodium currents in JME/CF15 cells, a cystic fibrosis airway epithelial cell line.

Am J Physiol Lung Cell Mol Physiol. 2004; 287: L928-935.

Vallet V, Chraibi A, Gaeggeler H P, Horisberger J D. & Rossier B C.

An epithelial serine protease activates the amiloride-sensitive sodium channel.

Nature. 1997; 389: 607-610.

1-22. (canceled)

23. A method of treating respiratory disease, comprising administration of a therapeutically effective amount of an amidino compound of the general formula: 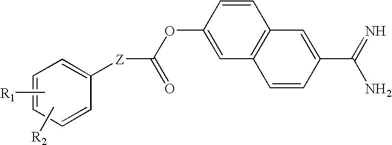
wherein;
Z represents —(CH₂)ₐ—, 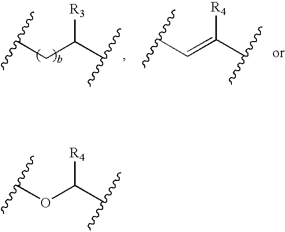
wherein a is 0, 1, 2 or 3, b is 0,1 or 2, R₃ is a straight or branched chain alkyl group of 1 to 4 carbon atoms or a cycloalkyl group of 3 to 6 carbon atoms, R₄ is a hydrogen atom or a straight or branched chain alkyl group of 1 to 4 carbon atoms;
R₁ and R₂, may be the same or different and represent each a hydrogen atom, a straight or branched chain alkyl group of 1 to 4 carbon atoms, —O—R₅, —S—R₅, —COOR₅, —COR₆, —O—COR₇, —NHCOR₇, 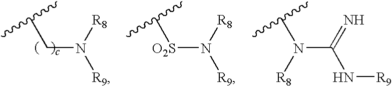
NO₂, CN, halogen, CF₃, methylenedioxy, or 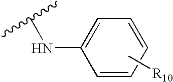
wherein c is 0, 1 or 2; R₅ is a hydrogen atom, linear or branched chain alkyl group of 1 to 4 carbon atoms, or benzyl group; R₆ is a hydrogen atom or straight or branched chain alkyl group of 1 to 4 carbon atoms; R₇ is a straight or branched chain alkyl group of 1 to 4 carbon atoms; R₈ and R₉, which may be the same or different, are each a hydrogen atom, straight or branched chain alkyl group of 1 to 4 carbon atoms, or amino radical protecting group; and R₁₀ is a hydrogen atom, methyl or CF₃;
to a patient in need thereof.

24. The method of claim 23 wherein Z represents a covalent bond.

25. The method of claim 23 wherein R₁ or R₂ represent hydrogen or a straight or branched alkyl group having from 1 to 4 carbon atoms.

26. The method of claim 23 wherein R₁ is hydrogen and R₂ is 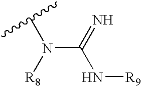
wherein R₈ and R₉ are hydrogen.

27. The method of claim 23, wherein the amidino compound is in the form of a pharmaceutically acceptable salt or ester.

28. The method according to claim 23, wherein the amidino compound is 6-amidino-2-napthyl 4-guanidinobenzoate.

29. The use according to claim 28, wherein the 6-amidino-2-napthyl 4-guanidinobenzoate is in the form of 6-amidino-2-napthyl 4-guanidinobenzoate dihydrochloride or 6-amidino-2-napthyl 4-guanidinobenzoate mesylate.

30. The method of claim 23, wherein the amidino compound is administered together with one or more pharmaceutically acceptable carriers or diluents.

31. The method of claim 23, wherein the medicament is administered as an aqueous solution.

32. The method according to claim 31, wherein the total chloride concentration administered is less than 10 mM.

33. The method according to claim 31, wherein the amidino compound is administered together with a pharmaceutically acceptable impermeant, monovalent anion with a concentration in the range of from 100 mM to 160 mM.

34. The method according to claim 33, wherein the pharmaceutically acceptable impermeant, monovalent anion is gluconate.

35. The method of claim 23 wherein the amidino compound is the sole active ingredient.

36. The method of claim 23 wherein the method additionally comprises coadministration of one or more other active agents selected from the group consisting of antibiotics, vaccines, decongestants (nasal or bronchial), mucolytic agents, rhDNase, non-steroidal antiinflamatory agents (NSAIDs), steroids, antiviral agents, elastase inhibitors, exofacial sodium channel blocking agents, gene therapy agents, chloride channel activators, mannitol, purinergic receptor agonists and bronchodilators.

37. The method of claim 36 wherein the medicament comprises only one additional active agent.

38. The method of claim 23, wherein the respiratory disease is characterised by poor mucociliary clearance or mucostasis.

39. The method of claim 23, wherein the respiratory disease is selected from the group consisting of cystic fibrosis (CF); bronchiectasis; chronic bronchitis; chronic obstructive pulmonary disease (COPD); rhino-sinusitis and otitis media.

40. The method of claim 23, wherein the respiratory disease is cystic fibrosis.

41. The method of claim 23, wherein the administration is to the respiratory system by the pulmonary route.

42. The method of claim 23, wherein the administration is by any one of intratracheal instillation, intratracheal delivery of liposomes, insufflation, nebulization, dry powder inhalation and aerosol inhalation.

43. The method of claim 23, wherein the administration is by dry powder inhalation or aerosol inhalation with a propellant selected from the group consisting of hydrofluroalkanes, chlorofluorocarbons, propane, nitrogen, or a mixture thereof.

44. The method of claim 23 wherein administration is as drops, by pipette or by syringe to the nasal epithelium, para-nasal sinuses, the Eustachian tube and middle ear, for irrigation of the para-nasal sinuses, or by solution directly applied to the middle ear via the external auditory meatus and canal.

45. A pharmaceutical composition comprising an amidino compound of the general formula: 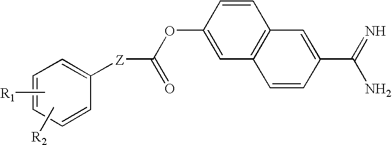
wherein;
Z represents —(CH₂)ₐ—, 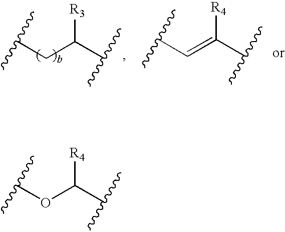
wherein a is 0, 1, 2 or 3, b is 0,1 or 2, R₃ is a straight or branched chain alkyl group of 1 to 4 carbon atoms or a cycloalkyl group of 3 to 6 carbon atoms, R₄ is a hydrogen atom or a straight or branched chain alkyl group of 1 to 4 carbon atoms;
R₁ and R₂, may be the same or different and represent each a hydrogen atom, a straight or branched chain alkyl group of 1 to 4 carbon atoms, —O—R₅, —S—R₅, —COOR₅, —COR₆, —O—COR₇, —NHCOR₇, 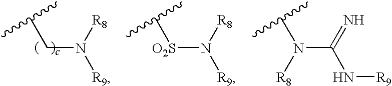
NO₂, CN, halogen, CF₃, methylenedioxy, or 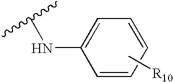
wherein c is 0, 1 or 2; R₅ is a hydrogen atom, linear or branched chain alkyl group of 1 to 4 carbon atoms, or benzyl group; R₆ is a hydrogen atom or straight or branched chain alkyl group of 1 to 4 carbon atoms; R₇ is a straight or branched chain alkyl group of 1 to 4 carbon atoms; R₈ and R₉, which may be the same or different, are each a hydrogen atom, straight or branched chain alkyl group of 1 to 4 carbon atoms, or amino radical protecting group; and R₁₀ is a hydrogen atom, methyl or CF₃;
formulated for administration to the respiratory system.

46.-63. (canceled)

64. An inhalation device loaded with the pharmaceutical composition of claim 45.

65.-67. (canceled)
